(19) United States
(12) Patent Application Publication
(10) Pub. No.: US 2006/0241525 A1
(43) Pub. Date: Oct. 26, 2006

(54) Therapeutic shockwave system with automatically controlled resumption of an interrupted treatment session

(76) Inventors: Markus Lanski, Nurnberg (DE); Christian Meinert, Marloffstein (DE)

Correspondence Address: SCHIFF HARDIN, LLP;PATENT DEPARTMENT, 6600 SEARS TOWER, CHICAGO, IL, 60606-6473, US

(21) Appl. No.: 11/365,178

(22) Filed: Mar. 1, 2006

(30) Foreign Application Priority Data
Mar. 1, 2005 (DE) 10 2005 009 907.6

(51) Int. Cl. A61H 1/00; A61B 8/00

(52) U.S. Cl. 601/2; 600/439

(57) ABSTRACT

A shockwave system has a shockwave source that generates shockwaves for treatment of a patient wherein, after an interruption of the treatment, the shockwave energy, starting from a predeterminable energy start value, is automatically, successively increased in predeterminable energy levels to a last energy end value applied and stored before the interruption of the treatment. Such a shockwave system enables an optimized resumption of a shockwave treatment after a treatment interruption.

### BACKGROUND OF THE INVENTION

1. Field of the Invention

The invention concerns a shockwave system with a shockwave source that generates shockwaves for a treatment of a patient.

2. Description of the Prior Art

Shockwave systems of the above type serve for treatment of the patient with extra-corporeal shockwaves, in the majority of cases for lithotripsy or pain therapy, in particular in extra-corporeal shockwave therapy (ESWT).

Lithotripsy is a therapeutic method to destroy a calculus (for example gallstone or kidney stone) located in the body of an organism without a surgical procedure, by using focused shockwaves. Both in lithotripsy and in extra-corporeal shockwave therapy, at the beginning the treatment is begun with low energy values in order to acclimate the patient to the treatment and the pain that may be associated with the treatment. This slow increase of the energy values that the operator manually implements is known as ramping. It is the goal to reach an optimally high energy level for the particular application in order to ensure an effective therapy. If the shockwave application to the patient is interrupted for various reasons for a time span of more than approximately one minute, the patient becomes “weaned” from the shockwave. Upon a resumption of the therapy, the patent therefore must be re-acclimated to the shockwave by ramping. This ramping is conventionally implemented manually be the operator until the last-applied energy value of the shockwave is reached.

### SUMMARY OF THE INVENTION

An object of the present invention is to provide a shockwave system of the type described above that enables an optimized resumption of the shockwave treatment after a treatment interruption.

This object is achieved in accordance with the invention by a shockwave system having a shockwave source that generates shockwaves for a treatment of a patient, wherein after an interruption of the treatment, the shockwave energy, starting from a predeterminable energy start value, is automatically, successively increased in predeterminable energy levels (stages) to a last energy end value that was applied and stored before the interruption of the treatment.

The shockwave system according to the invention enables an optimized resumption of the shockwave treatment in patients after a treatment interruption. Moreover, the energy levels can be calibrated significantly more finely than in the case of a manual increase. For this purpose, with each individual shockwave the high voltage that determines the energy value is increased by only a very small amount. With this changes are possible that correspond to only a fraction of a conventional (manual) increase of the energy values. A very gentle (conservative) acclimation of the patient to the last applied energy end value is thereby possible since a spiking or jumping increase of the energy values as well as a severe increase of the pain sensation that is associated therewith is reliably avoided.

Since the increase to the last energy end value applied before the interruption of the treatment ensues automatically, only the parameters for the energy levels must be predetermined by the operator (this can, for example, ensue in the startup of the shockwave system), and the shockwave system can be started at a predeterminable energy start value. Generally, a further intervention by the operator is necessary until reaching the energy end value.

The increase of the shockwave energy can be parameterized in multiple ways in the context of the invention. For example, the predeterminable energy start value at which the treatment is resumed after an interruption can be constant, in particular zero.

According to a further embodiment of the invention, the energy start value is predeterminable dependent on the energy end value applied before the last interruption of the treatment. In the event that a certain acclimation to the shockwaves has already been reached with a patient, a higher energy start value (entrance value) with than the minimal energy start value can be used at this re-starting time. The time until reaching the energy end value applied before the interruption of the treatment thus can be distinctly shortened.

In a further embodiment, the successive increase of the shockwave energy can be implemented in fine stages, such that the increase essentially exhibits a constant slope. The more finely staged that the increase of the shockwave energy is implemented, the more gentle the acclimation of the patient to the last energy end value applied before interruption of the treatment.

This slope (curve) of the increase of the shockwave energy can essentially form a straight line, but other curves deviating from a straight line are also possible for the increase of the shockwave energy.

The curve of the increase of the shockwave energy can be defined by a predeterminable function. This function can be predetermined, for example, dependent on the energy end value applied before the last interruption of the treatment and/or dependent on a function of the curve of a previous increase of the shockwave energy.

In another embodiment of the shockwave system according to the invention, the automatic increase of the shockwave energy automatically terminates given a manual intervention, and the system automatically switches into a manual mode.

In an embodiment of the invention the shockwave energy can be manually adjusted to a new energy end value after reaching the energy end value applied before the last interruption of the treatment.

In particularly user-friendly embodiments of the inventive shockwave system, the values and/or the intermediate values of the energy levels can be displayed, or the curve of the increase of the shockwave energy, and/or curve of the intermediate values, can be graphically represented.

Through the aforementioned parameterization possibilities, the curve of the increase of the shockwave energy can be adapted in a simple manner to different treatment methods and to the different pain sensitivities of individual patients. User-specific application variants can also be realized in a simple manner with this. This parameterization can ensue both once via a service setting at the first startup or, respectively, given maintenance jobs and by the user himself.

### DESCRIPTION OF THE DRAWINGS

FIG. 1 shows a curve of the increase of the shockwave energy according to a first parameterization in accordance with the invention.

FIG. 2 shows a curve of the increase of the shockwave energy according to a second parameterization in accordance with the invention.

### DESCRIPTION OF THE PREFERRED EMBODIMENTS

The system in accordance with the invention for automatic resumption of an interrupted shockwave treatment can be embodied in the high voltage supply and/or a control unit therefor for a shockwave source of the type described in U.S. Pat. No. 4,674,505 or U.S. Pat. No. 4,905,675. The teachings of both of those patents are incorporated herein by reference.

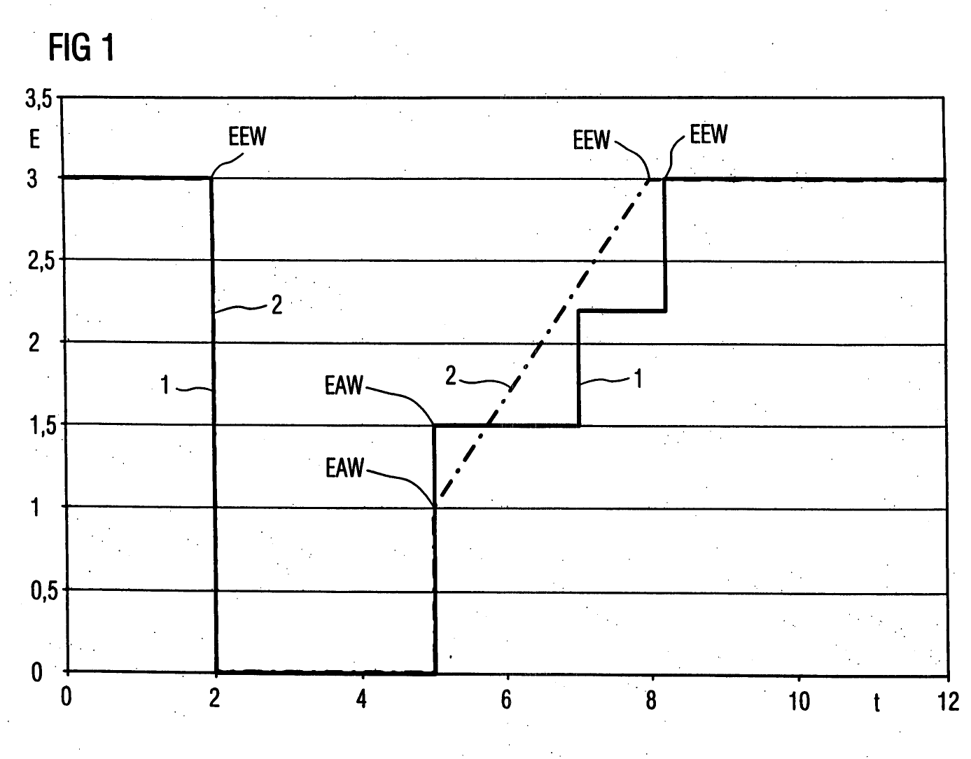

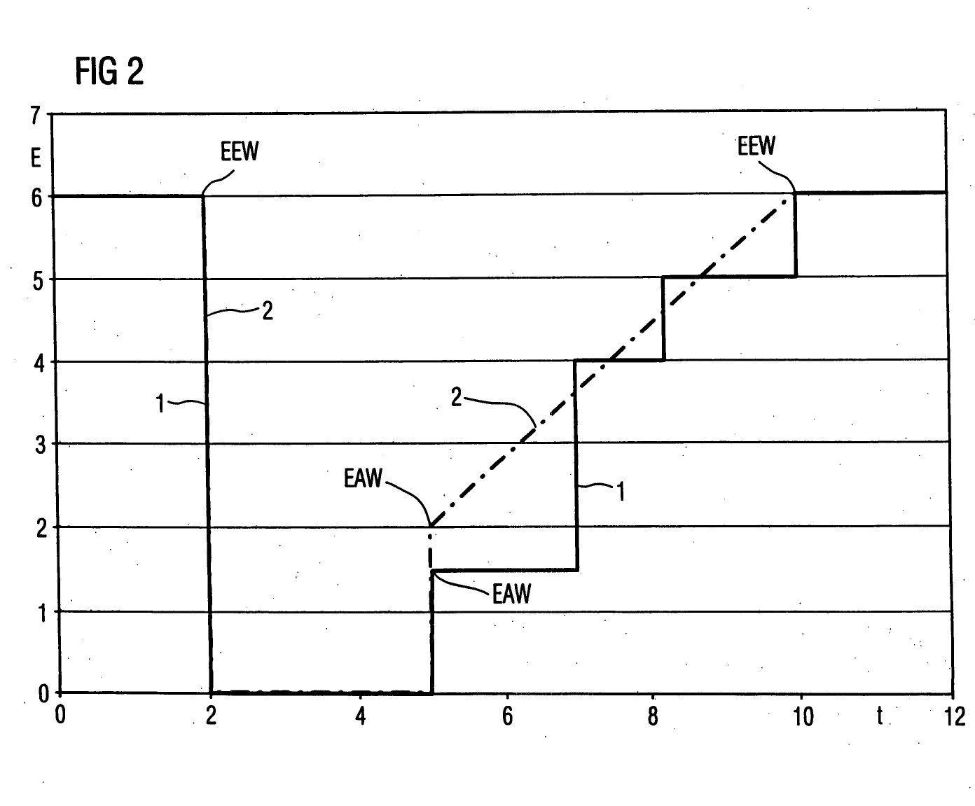

In FIGS. 1 and 2, the time t is plotted as a dimensionless quantity on the abscissa. The shockwave energy E is plotted as a dimensionless quantity on the ordinate.

In FIGS. 1 and 2, an exemplary curve of a manual increase of the shockwave energy E is designated 1. A curve of an automatic increase of the shockwave energy E, which can be achieved with the inventive shockwave system, is designated 2.

The treatment shown in FIG. 1 is manually interrupted at an energy end value EEW=3 at t=2. The treatment with an energy start value EAW=1.5 is continued at t=5.

The energy levels exhibit different heights as well as different amplitudes. At approximately t=8.1, the energy end value applied before the last interruption of the treatment is reached with the energy end value EEW=3.

Relative to the manual increase of the shockwave energy, the automatic increase of the shockwave energy in the inventive shockwave system ensues successively and in fine stages, such that the increase essentially exhibits a constant slope. In the shown exemplary embodiment, the slope of the increase of the shockwave energy essentially forms a straight line.

The selected curve begins with an energy start value that, at point in time t=5, lies at EAW=1 and, for example, exhibits a slope of 0.67. The energy end value EEW=3 is, for example, reached at t=8.1.

The treatment shown in FIG. 2 is manually interrupted at an energy end value EEW=6 at t=2. The treatment is continued at t=5 with an energy start value EAW=1.5.

The energy levels exhibit different heights as well as different amplitudes. At t=10, the energy end value applied before the last interruption of the treatment is reached with the energy end value EEW=6.

Relative to the manual increase of the shockwave energy, the automatic increase of the shockwave energy in turn ensues successively and in fine stages, such that the increase essentially exhibits a constant slope. In the shown exemplary embodiment, the slope of the increase of the shockwave energy likewise essentially forms a straight line.

The selected curve begins with an energy start value that, at point in time t=5, lies at EAW=2 and, for example, exhibits a slope of 0.8. The energy end value EEW=6 is reached at t=10.

From the explanation of both (non-limiting) exemplary embodiments of the invention, it is clear that an optimized resumption of the shockwave treatment after a treatment interruption is possible in a simple manner with the inventive shockwave system.

Although modifications and changes may be suggested by those skilled in the art, it is the intention of the inventors to embody within the patent warranted hereon all changes and modifications as reasonably and properly come within the scope of their contribution to the art.

We claim as our invention:

1. A shockwave system comprising:
a shockwave source that generates shockwaves, having an energy content, for treating a patient;
a control unit that operates said shockwave source to apply said energy to a patient during said treatment and, after an interruption of said treatment, said control unit automatically successively increasing said energy, starting from a predetermined energy value, in steps up to a last energy end value applied and stored before said interruption of said treatment.

2. A shockwave system as claimed in claim 1, whereby the energy start value is constant, in particular zero.

3. A shockwave system as claimed in claim 1, whereby the energy start value can be predetermined dependent on the energy end value applied in the last treatment.

4. A shockwave system as claimed in claim 1 wherein said control unit sets said energy start value to a predetermined value dependent on said energy and value applied before a last interruption of said treatment.

5. A shockwave system as claimed in claim 1 wherein said control unit successively increases said energy in fine steps so that an increase in said energy exhibits a substantially constant step.

6. A shockwave system as claimed in claim 5 wherein said control unit sets a curve for said increase that substantially forms a straight line.

7. A shockwave system as claimed in claim 5 wherein said control unit sets a curve for the increase of said energy according to a predetermined function.

8. A shockwave system as claimed in claim 6 wherein said control unit predetermines said function dependent on an energy end value applied in an immediately preceding treatment.

9. A shockwave system as claimed in claim 1 wherein said control unit automatically deactivates said automatic increase of said energy upon a manual intervention, and then switches into a manual mode for operating said shockwave source.

10. A shockwave system as claimed in claim 1 wherein said control unit allows manual adjustment of said energy to a new energy end value after reaching said energy and value applied in a preceding treatment.

11. A shockwave system as claimed in claim 1 wherein said control unit comprises a display at which at least one of said energy start value, said energy end value and values therebetween are displayed.

12. A shockwave system as claimed in claim 1 wherein said control unit comprises a display at which a curve of the increase of said energy is graphically displayed.
